(19) United States
(12) Patent Application Publication
(10) Pub. No.: US 2026/0245737 A1
(43) Pub. Date: Aug. 20, 2026

(54) METHOD AND SYSTEM FOR ESTIMATION OF THREE-DIMENSIONAL STRUCTURES USING TWO-DIMENSIONAL IMAGES

(71) Applicant: The Chinese University of Hong Kong, Hong Kong SAR (CN)

(72) Inventors: Benny Chung-Ying ZEE, Hong Kong SAR (CN); Jack Jock-Wai LEE, Hong Kong SAR (CN); Chuying SHI, Hong Kong SAR (CN)

(21) Appl. No.: 19/489,570

(22) PCT Filed: Jun. 5, 2024
(86) PCT No.: PCT/CN2024/097437

§ 371 Date: Dec. 2, 2025

Related U.S. Application Data
(60) Provisional application No. 63/471,706, filed on Jun. 7, 2023.

(51) Int. Cl. G16H 50/30; G06N 3/08; G06T 7/00; G06T 7/11

(52) U.S. Cl. CPC G16H 50/30; G06N 3/08; G06T 7/0012; G06T 7/11; G06T 2207/10101; G06T 2207/20081; G06T 2207/20084; G06T 2207/30041

(57) ABSTRACT

A method of estimating optical coherence tomography (OCT) parameters includes obtaining a retinal fundus image and segmenting the retinal fundus image to produce a region of interest 2D image. The method also includes inputting the region of interest 2D image into a neural network and generating estimated OCT parameters using the neural network.

### CROSS-REFERENCES TO RELATED APPLICATIONS

This application claims priority to U.S. Provisional Patent Application No. 63/471,706, filed on Jun. 7, 2023, entitled “Method and System for Estimation of Three-Dimensional Structures Using Two-Dimensional Images,” the disclosure of which is hereby incorporated by reference in its entirety for all purposes.

### TECHNICAL FIELD

The present disclosure relates to image processing, particularly to a method and a system of estimating optical coherence tomography parameters.

### BACKGROUND

Optical coherence tomography (OCT) devices are non-contact, non-invasive, and objective structural imaging devices for cross-sectional and three-dimensional (3D) viewing of the macula and optic nerve head. For several years, OCT has been commercially available and accepted as a clinical standard within ophthalmology for the diagnosis of retinal diseases. Moreover, OCT has been useful for 3D imaging in the areas of neuro-ophthalmology and neurodegeneration. Accordingly, OCT has become a valuable tool for diagnosing, monitoring, and predicting the prognosis of many diseases.

However, the OCT instrument is bulky, operation of the OCT instrument is time-consuming and difficult for some operators, and the procedure can be difficult to tolerate for some patients. Thus, OCT has limitations in its application and is not commonly used in a general medical office or a community screening setting. Thus, there is a need in the art for improved methods and systems related to structural imaging.

### SUMMARY

Embodiments of the present invention relate to image processing. More particularly, embodiments of the present invention provide methods and systems for estimating 3D OCT parameters using 2D fundus camera images. In a specific embodiment, a machine-learning system is utilized to estimate one or more OCT parameters using a 2D image as an input. The present invention is applicable to 3D parameter estimation in applications outside optometry and ophthalmology including other image processing applications.

As discussed above, OCT devices provide 3D images of the eye. Fundus cameras can be used to acquire retinal images (e.g., non-mydriatic retinal images) at low-cost since fundus cameras are portable, quick and straightforward to operate, and data interpretation is readily achieved. The procedure of fundus camera imaging is also well accepted and tolerated by patients and operators can easily use it. In addition, studies have verified it is practical and effective in the community setting.

Using machine-learning methods and systems, embodiments of the present invention apply artificial intelligence (AI) approaches that can, based for instance, on hidden features present in fundus images, estimate comprehensive OCT parameters using non-mydriatic retinal images. These OCT parameters include retinal nerve fiber layer (RNFL) thickness; optic nerve head (ONH) parameters: rim area, disc area, average cup-to-disc-ratio (C/D), vertical C/D, and cup volume; and average and minimum ganglion cell-inner plexiform layer (GCIPL) thickness.

As described herein, methods and systems operable to estimate comprehensive OCT parameters based on color fundus retinal images are provided. The results demonstrate that embodiments of the present invention can be a convenient, cost-effective, and accurate tool to screen, diagnose, and monitor glaucoma and other diseases that can be assessed by OCT, especially in the tele-medicine system and community setting where the OCT is not available.

Numerous benefits are achieved by way of the present disclosure over conventional techniques. For example, embodiments of the present invention provide a convenient, cost-effective, and accurate tool to screen for, assist in the diagnosis of, indicate the severity of, and monitor the progression of diseases, especially in the tele-medicine system and community setting where the OCT may not be available. Using embodiments of the present invention, the advent of AI techniques can be combined with tele-technology to create greater community healthcare services. For example, in third-world settings where residents in rural areas have limited access to proper healthcare, the cost-effective and practical tool for screening described herein is of significant relevance in facilitating prompt referral to a clinician and greatly assists in the subsequent diagnosis, treatment, and follow-up to reduce the medical burden. These and other embodiments of the disclosure, along with many of its advantages and features, are described in more detail in conjunction with the text below and corresponding figures.

Using any of the methods described herein, a patient who has been identified as suffering from or may be at increased risk of developing one or more neuro-ophthalmology diseases such as glaucoma, nonarteritic ischemic optic neuropathy (NAION), Leber's hereditary optic neuropathy (LHON), compressive optic neuropathies, disc swelling, idiopathic intracranial hypertension, or neuro-myelitis optica (NMO) spectrum disorder may be subject to additional testing using conventional diagnostic and monitoring methods such as tonometry (measuring intraocular pressure), eye dilation and imaging tests (detecting optic nerve damage), visual field tests (detecting areas of vision loss), pachymetry (measuring corneal thickness), and gonioscopy (inspecting the drainage angle). As deemed appropriate by the attending physician, the patient may further receive early treatment in order to prevent or delay the onset of symptoms, reduce symptom severity, and/or prevent the development of more advanced and damaging symptoms. For example, eye drops containing the following medications may be prescribed and administered to the patient: prostaglandins (to reduce eye pressure), beta blockers (to reduce fluid production/build-up in the eyes and thus reduce eye pressure), alpha-adrenergic agonists (to reduce fluid production in the eye and to increase fluid outflow from the eye), carbonic anhydrase inhibitors (to reduce fluid production in the eye), rho kinase inhibitor (to reduce eye pressure), and miotic or cholinergic agents (to increase fluid outflow from the eye).

Similarly, a patient who has been so deemed to suffer from or may be at increased risk of developing one or more neurodegeneration diseases such as Alzheimer's disease (AD), mild cognitive impairment (MCI), Parkinson's disease, dementia, or Huntington's disease may be subject to additional diagnostic examination to provide further confirmatory information (for example, by brain imaging via CT scan or other imaging techniques to show excessive loss of brain volume, or by testing cognitive capability to show an accelerated decline), and suitable therapeutic or prophylactic regimens may be ordered by physicians or other medical professionals to treat the patient, to manage/alleviate the ongoing symptoms, or to delay the future onset of the disease. The U.S. Food and Drug Administration (FDA) has approved a number of cholinesterase inhibitors, including donepezil (Aricept™, acholinesterase inhibitor approved to treat all stages of AD, including moderate to severe AD), rivastigmine (Exelon™, approved to treat mild to moderate AD), galantamine (Razadyne™, mild to moderate patients) and memantine (Namenda™). Any one or more of these drugs can be prescribed for treating patients who have been diagnosed with MCI or AD in accordance with the methods described herein. Another possible treatment is administration of trazodone, which is currently approved for use as an antidepressant and has been reported as an effective agent for ameliorating MCI or AD symptoms.

For a patient who is deemed to suffer from or is at increased risk of developing conditions such as hypertensive retinopathy, high blood pressure, intracranial tumors that affect the optic pathway, sellar and parasellar lesions, axonal degeneration in eyes of patients with multiple sclerosis (MS), diabetic peripheral neuropathy (DPN), Type 1 diabetes, or MRI-measured brain atrophy or spinal cord lesions, additional conventional tests may be performed for the purpose of regular monitoring and/or diagnosis of a pertinent disease. As deemed appropriate, blood pressure-lowering medicaments (such as amlodipine, carvedilol, furosemide, etc.) and cholesterol-lowering medications as well as insulin may be prescribed and administered to the patient.

### BRIEF DESCRIPTION OF DRAWINGS

FIG. 1 is a simplified schematic diagram illustrating a system for estimating 3D characteristics based on 2D images according to an embodiment of the present invention.

FIG. 2 is a simplified flowchart illustrating a method of estimating OCT parameters using color images according to an embodiment of the present invention.

FIG. 3 is a simplified flowchart illustrating a method of training a neural network according to an embodiment of the present invention.

FIG. 4 is a simplified flowchart illustrating a method of estimating 3D characteristics using a trained neural network according to an embodiment of the present invention.

FIG. 5A is a segmented region of interest of an optic disc for a normal eye according to an embodiment of the present invention.

FIG. 5B is a segmented region of interest of an optic disc for an eye with glaucomatous optic neuropathy (GON) according to an embodiment of the present invention.

FIG. 5C is a segmented region of interest of a macula for a normal eye according to an embodiment of the present invention.

FIG. 5D is a segmented region of interest of a macula for an eye with GON according to an embodiment of the present invention.

FIGS. 6A-6H are box plots of OCT parameters according to an embodiment of the present invention.

FIGS. 7A-7H are violin plots demonstrating true and estimated OCT parameters according to an embodiment of the present invention.

FIG. 8 is a simplified schematic diagram illustrating an OCT parameter estimation system according to an embodiment of the present invention.

### DETAILED DESCRIPTION OF EMBODIMENTS

Embodiments of the present invention relate to image processing. More particularly, embodiments of the present invention provide methods and systems for estimating 3D OCT parameters using 2D fundus camera images. In a specific embodiment, a machine-learning system is utilized to estimate one or more OCT parameters using a 2D image as an input. The present invention is applicable to 3D parameter estimation in applications outside optometry and ophthalmology including other image processing applications.

As described herein, embodiments of the present invention provide methods and systems that can be utilized in relation to various diseases correlated with OCT parameters. Embodiments provide a convenient, cost-effective, and accurate tool to screen, assist clinicians during diagnosis, indicate the severity of, and monitor the progression of the diseases, especially in the tele-medicine system and community setting where the OCT may not be available.

OCT is an ocular imaging technique that can provide high-resolution, cross-sectional (i.e., 3D) images. Currently, as a clinical tool, OCT is particularly useful for the structural measurement of peripapillary RNFL thickness; ONH volumetric analysis including disc area, rim area, average cup-to-disc ratio, vertical cup-to-disc ratio, and cup volume; and macular anatomy including GCIPL thickness.

OCT measurements are useful for identifying the integrity of visual pathways in intracranial lesions, neuro-ophthalmology, and the neurodegeneration process of visual pathways. For several years, OCT has been commercially available and accepted as a clinical standard within ophthalmology for the diagnosis of retinal diseases. Moreover, the RNFL thickness, ONH parameters, and GCIPL thickness are especially useful in neuro-ophthalmology. OCT has become a valuable tool for diagnosis, disease monitoring, and predicting prognoses in neuro-ophthalmology such as nonarteritic ischemic optic neuropathy (NAION), and Leber's hereditary optic neuropathy (LHON), compressive optic neuropathies, disc swelling, idiopathic intracranial hypertension, and neuro-myelitis optica (NMO) spectrum disorder.

Recently, OCT was found useful in detecting neurodegeneration such as Alzheimer's disease (AD), mild cognitive impairment (MCI), and Parkinson's disease. The RNFL and GCIPL thickness were reported to be associated with hypertensive retinopathy and blood pressure, stroke risk (e.g., ischemic stroke or hemorrhagic stroke), intracranial tumors that affect the optic pathway, sellar and parasellar lesions, axonal degeneration in the eyes of patients with multiple sclerosis (MS), diabetic peripheral neuropathy (DPN), and other surrogate markers used to monitor disease activity such as MRI-measured brain atrophy and spinal cord lesions. RNFL thinning is also associated with dementia, Huntington's disease, the severity of papilledema, autism, attention-deficit/hyperactivity disorder (ADHD), autism spectrum disorder (ASD), and mental health diagnoses including depression. Additionally, a study has demonstrated the loss of the RNFL and GCIPL thickness in patients with Type 1 diabetes without retinopathy compared with healthy controls. Thus, embodiments of the present invention provide insight into the retinal nerve fiber layer of the eye, which makes some embodiments applicable, not only to the detection and treatment of glaucoma, but to a variety of systemic diseases, including those mentioned above.

Automatic classification using AI methods has been used to detect various retinopathies and other diseases and achieved good performance on fundus images. However, simple classification cannot provide adequate information for the screening, diagnosis, and follow-up of diseases. Moreover, AI methods such as deep learning (DL) generally lack interpretability in relation to disease detection. Embodiments of the present invention utilize a DL model that is trained to learn the underlying features in data from multiple layers of networks, thereby providing quantitative assessments of the OCT parameters used by clinicians to diagnose, indicate the severity of, and monitor the progression of various diseases correlated with OCT parameters. Additionally, embodiments of the present invention increase the explainability and improve the classification performance of automatic retinal image analysis methods.

As described herein, embodiments of the present invention utilize color fundus retinal images (e.g., obtained using an automated retinal image analysis (ARIA) method, to objectively estimate comprehensive OCT parameters that have been measured by OCT optic disc scans and macular scans, including ONH parameters (disc area, rim area, average C/D ratio, vertical C/D ratio, and cup volume), average RNFL thickness, and average and minimum GCIPL thickness. To better estimate the OCT parameters at different levels, patients were selected with/without glaucomatous optic neuropathy (GON), which is a typical neuropathy characterized by changes in RNFL thickness, ONH parameters, and GCIPL thickness.

From the perspective of medicine, embodiments of the present invention predict the continuous parameters of OCT scans, including 3D parameters, based on 2D non-mydriatic color fundus retinal images. Some embodiments do not focus on a single OCT parameter, but on eight parameters related to the optic disc and macular area, which can provide comprehensive retinal information for the better estimation of diseases. Thirdly, images captured using non-mydriatic photography can be utilized, which is more convenient, feasible, and tolerable compared with mydriatic images. Although non-mydriatic retinal images may pose challenges in training due to the low pixel count and relative blurriness, embodiments of the present invention still achieve excellent performance. Although mydriatic retinal images can be utilized and embodiments of the present invention are not limited to the use of non-mydriatic images, non-mydriatic images were used since tele-medicine, as a practical, convenient and beneficial solution for patients and the health care system, is more commonly performed without dilating agents and uses non-mydriatic images.

From the methodological perspective, embodiments of the present invention have the ability of explanation and avoid the “black box” problem corresponding to some DL networks. Moreover, the methods and systems described herein are more accurate than traditional machine-learning methods and can be regularly refined by retraining the DL network.

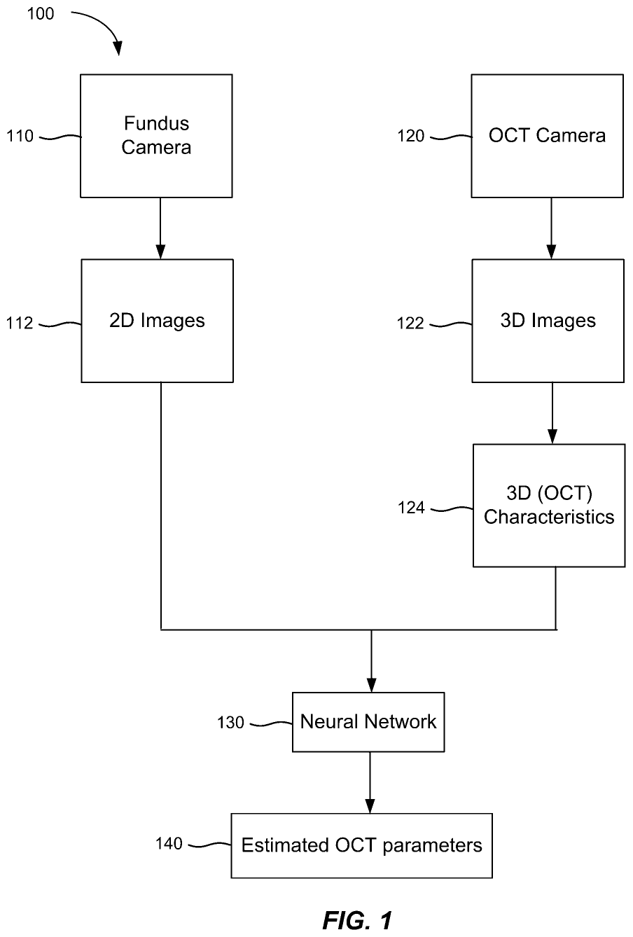

FIG. 1 is a simplified schematic diagram illustrating a system for estimating 3D characteristics based on 2D images according to an embodiment of the present invention. The system 100 includes a fundus camera 110 that is operable to capture 2D images 112, for example, non-mydriatic RGB images of a patient's retina. The 2D images 112 are generally segmented based on regions of interest as discussed more fully below in relation to the ARIA method. The system 100 also includes an OCT camera 120 that is operable to capture 3D images 122 and generate 3D (OCT) characteristics 124 of the retina. These 3D (OCT) characteristics 124 can include characteristics corresponding to the optic disc area as well as the macula.

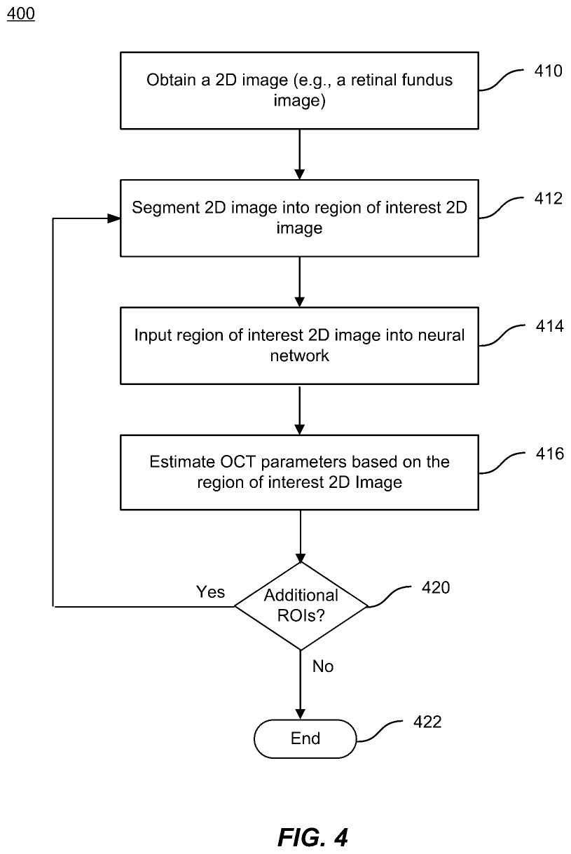

The system 100 further includes a neural network 130 that is trained using the 2D images 112 (e.g., automatically segmented 2D images that are segmented into one or more regions of interest) and the 3D (OCT) characteristics 124. During operation as described more fully in relation to FIG. 4, the neural network 130 can receive a 2D image as an input and output estimated OCT parameters 140, i.e., 3D characteristics based on 2D images.

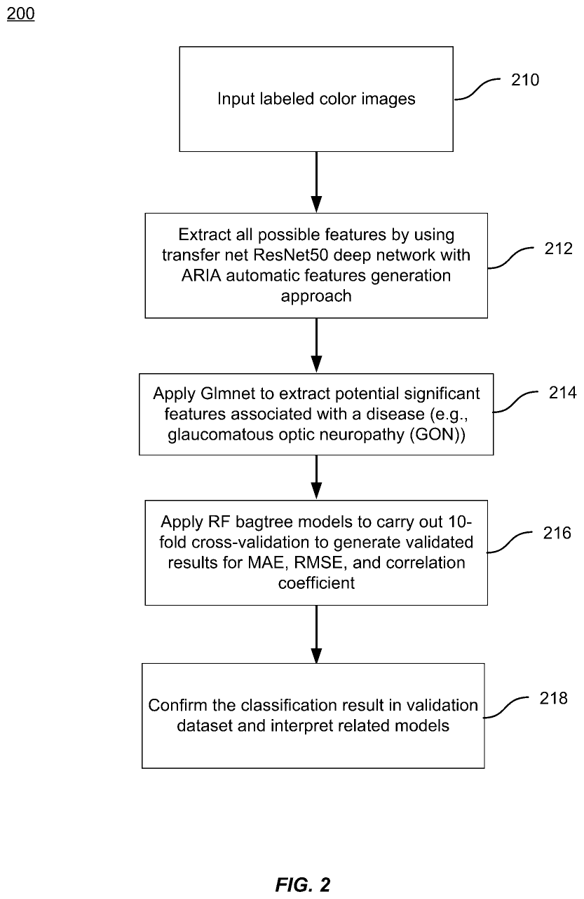

FIG. 2 is a simplified flowchart illustrating a method of estimating OCT parameters using color images according to an embodiment of the present invention. The process illustrated in FIG. 2 can be referred to as a model generation process and shares common elements with the system 100 illustrated in FIG. 1. Accordingly, the description provided in relation to FIG. 1 is applicable to FIG. 2 as appropriate. As illustrated by the method 200 in FIG. 2, labeled color (e.g., RGB) images (e.g., non-mydriatic color images obtained using a fundus camera) are received as inputs (210) and features are generated by applying a transfer net ResNet-50 deep network with retinal images as input and features generated at the layer of “fc1000_softmax” as output, and the ARIA automatic features generation approach based on pixels associated with a disease, for example, GON (212). Training of the DL can utilize OCT parameters corresponding to the color images (i.e., the color images can be labeled using OCT parameters and the labeled images can be utilized as inputs for the training process) as discussed herein.

Although a transfer net ResNet-50 DL is illustrated in FIG. 2, embodiments of the present invention can utilize other DL networks or machine-learning models. Examples of machine-learning models include a random forest model, including deep random forests, neural networks, including recurrent neural networks and convolutional neural networks, graph-based convolutional neural networks, quaternion neural networks, restricted Boltzmann machines, recurrent tensor networks, and gradient boosted trees. Thus, a variety of models including deep learning models (e.g., neural networks having many layers), random forest models, decision trees, a support vector machine (SVM), neural networks, and K-nearest neighbors (KNN), including the use of boosting (i.e., AdaBoost) are included within the scope of the present invention.

Then, the Glmnet approach was applied to select subsets of the feature, e.g., the most important subsets of features, that were highly associated with a disease, for example, GON (214). This process, which can be considered as a statistical process, reduces the noise resulting from less significant features as well as the computational complexity. Although some embodiments are discussed in the context of glaucoma, as discussed more fully herein, embodiments of the present invention are applicable to a variety of other eye conditions, eye diseases, health conditions, and/or diseases that are correlated with OCT parameters. To avoid over-fitting problems, a validation method, for example, 10-fold cross-validation with random forest (RF) bagtree models, was applied to generate more robust results (216). Although 10-fold cross-validation with random forest (RF) bagtree models is illustrated in FIG. 2, this particular validation process is not required and other validation processes are included within the scope of the present invention. Moreover, in some embodiments, validation is optional. Finally, the method 200 includes confirming the prediction performance of the RF models in the validation dataset (218). In some embodiments, this confirmation process is also optional.

It should be appreciated that the specific steps illustrated in FIG. 2 provide a particular method of estimating OCT parameters using color images according to an embodiment of the present invention. Other sequences of steps may also be performed according to alternative embodiments. For example, alternative embodiments of the present invention may perform the steps outlined above in a different order. Moreover, the individual steps illustrated in FIG. 2 may include multiple sub-steps that may be performed in various sequences as appropriate to the individual step. Furthermore, additional steps may be added or removed depending on the particular applications. One of ordinary skill in the art would recognize many variations, modifications, and alternatives.

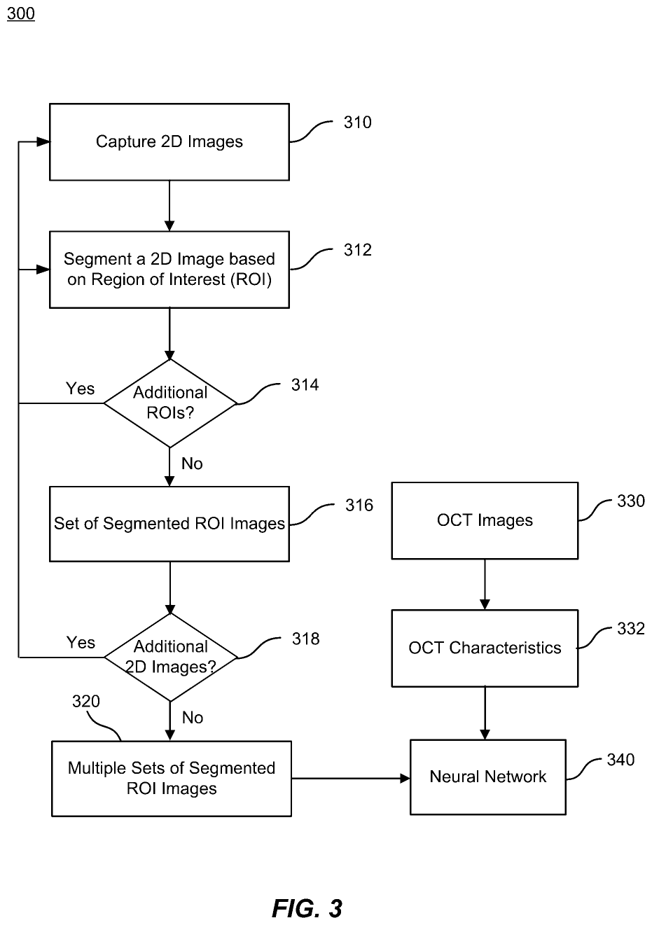

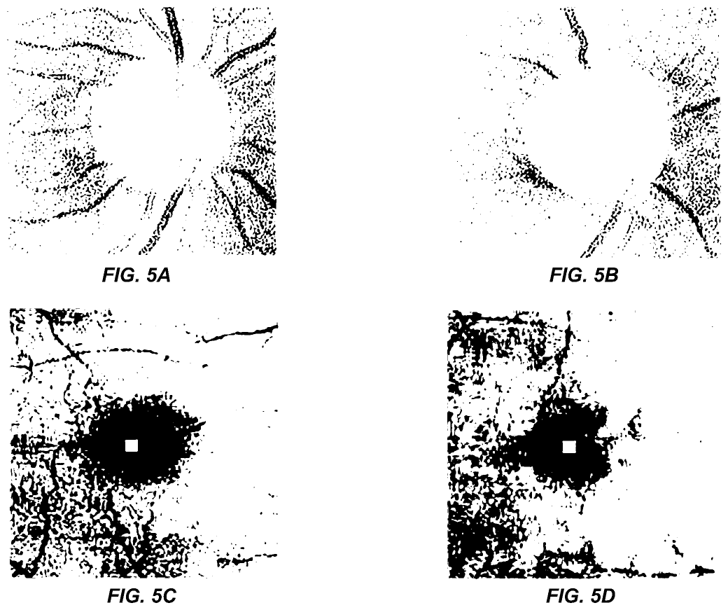

FIG. 3 is a simplified flowchart illustrating a method of training a neural network according to an embodiment of the present invention. The method 300 includes capturing a plurality of 2D images (310). The 2D images can be color (e.g., RGB) retinal images captured using a color fundus camera. The method 300 also includes segmenting a first 2D image based on a region of interest (ROI) (312). As discussed in relation to FIGS. 5A-5D below, the ROI can be the optic disc area, the macula of the retina, or other ROIs. Additionally, in some embodiments, the ROI includes the entirety of the retinal image or substantially the entirety of the retinal image. In these embodiments, the segmentation process illustrated by process 312 is substantially bypassed to provide the original retinal image for subsequent processing.

The method 300 further includes determining if there are additional ROIs (314). As an example, if a first segmentation process produces an optic disc image as the first ROI, an additional ROI could be the macula and the original 2D image could be segmented at process 312 to produce a macula image. Once the desired ROIs have been utilized during the segmentation process, a set of segmented ROI images are produced (316). For the remaining 2D images that are available, processes 312 and 314 are repeated using the remaining 2D images until multiple sets of segmented ROI images are produced (320). If not additional 2D image remain (318), then the multiple sets of segmented ROI images are provided as inputs to a neural network (340).

In order to train the neural network, in addition to the multiple sets of segmented ROI images (320), OCT images corresponding to the 2D images are captured (330) and OCT characteristics are determined based on the OCT images (332). In some embodiments, the OCT characteristics are utilized to label the 2D images contained in the multiple sets of segmented ROI images. Thus, embodiments pair 2D images with a corresponding OCT image in order to provide training data for the neural network. Thus, the neural network is trained using the multiple sets of segmented ROI images and the OCT characteristics as inputs (340).

It should be appreciated that the specific steps illustrated in FIG. 3 provide a particular method of training a neural network according to an embodiment of the present invention. Other sequences of steps may also be performed according to alternative embodiments. For example, alternative embodiments of the present invention may perform the steps outlined above in a different order. Moreover, the individual steps illustrated in FIG. 3 may include multiple sub-steps that may be performed in various sequences as appropriate to the individual step. Furthermore, additional steps may be added or removed depending on the particular applications. One of ordinary skill in the art would recognize many variations, modifications, and alternatives.

FIG. 4 is a simplified flowchart illustrating a method of estimating OCT parameters using a trained neural network according to an embodiment of the present invention. The method 400 includes obtaining a 2D image (e.g., a retinal fundus image) (410) and segmenting the 2D image to produce a region of interest 2D image (412). Segmentation of the 2D image can be performed using the ARIA method as discussed above.

The method also includes inputting the region of interest 2D image into a neural network (414) and estimating OCT parameters using the neural network (416). If additional region of interest 2D images are available (420), then processes 412, 414, and 416 are repeated to estimate additional OCT parameters. Once all the region of interest 2D images have been processed, the method ends (422).

The OCT parameters can include ONH parameters such as rim area, disc area, average C/D, vertical C/D, or cup volume. For these ONH parameters, the region of interest 2D image is an optic disc image and the ONH parameters are derived from an OCT optic disc scan. In other embodiments, the OCT parameters include retinal nerve fiber layer (RNFL) thickness, for example, an average RNFL thickness. For this OCT parameter, the region of interest 2D image includes an optic disc image and the RNFL thickness is derived from an OCT optic disc scan.

In some embodiments, the OCT parameters include GCIPL thickness. The GCIPL thickness can be an average GCIPL thickness or a minimum GCIPL thickness. In this case, the region of interest 2D image includes a macula image and the GCIPL thickness is derived from an OCT macular scan.

The method can further include recommending a medical procedure based on the estimated OCT parameters or obtaining one or more additional 2D images, including color retinal fundus images, based on the estimated OCT parameters.

Although FIG. 4 has been discussed in relation to the use of a retinal fundus image as an example of a 2D image and OCT parameters as examples of 3D characteristics that are determined based on the region of interest 2D image formed after segmentation of the retinal fundus image, it will be appreciated that the embodiments described herein are applicable to a variety of 2D images and a variety of 3D characteristics. Thus, although the method 400 is discussed in the context of a specific 2D image (e.g., a retinal fundus image) and a specific set of 3D characteristics (e.g., OCT parameters), it will be appreciated that the method is applicable to other 2D images and other 3D characteristics. Thus, applicability is not limited to ophthalmic applications or medical applications, but other applications in which 3D characteristics can be estimated based on 2D images. One of ordinary skill in the art would recognize many variations, modifications, and alternatives.

It should be appreciated that the specific steps illustrated in FIG. 4 provide a particular method of estimating OCT parameters using a trained neural network according to an embodiment of the present invention. Other sequences of steps may also be performed according to alternative embodiments. For example, alternative embodiments of the present invention may perform the steps outlined above in a different order. Moreover, the individual steps illustrated in FIG. 4 may include multiple sub-steps that may be performed in various sequences as appropriate to the individual step. Furthermore, additional steps may be added or removed depending on the particular applications. One of ordinary skill in the art would recognize many variations, modifications, and alternatives.

To generate exact optic disc features and macular features, embodiments of the present invention localize and segment the optic disc and macula by setting the ROI as the rectangular area surrounding the optic disc or macula, respectively. To compute the retinal data discussed herein, the ARIA method developed to acquire and analyze retinal images was used. The ARIA method is discussed in additional detail in U.S. Pat. No. 8,787,638, the disclosure of which is hereby incorporated by reference in its entirety for all purposes. Approaches including Haralick texture features analysis, fractal analysis, and a set of modified pre-trained deep networks (i.e., modified transfer network resnet50) were used to create the highly related pixels of features.

FIG. 5A is a segmented region of interest of an optic disc for a normal eye according to an embodiment of the present invention. FIG. 5B is a segmented region of interest of an optic disc for an eye with glaucomatous optic neuropathy (GON) according to an embodiment of the present invention. After an image of the retina is captured using, for example, a color fundus camera, the image is segmented into one or more regions of interest. In FIG. 5A, an ROI of the optic disc area is identified and the image is segmented, for example, automatically, using the ARIA method to produce the image illustrated in FIG. 5A. The coordinates of the ROI in the original image can also be determined.

FIG. 5C is a segmented region of interest of a macula for a normal eye according to an embodiment of the present invention. FIG. 5D is a segmented region of interest of a macula for an eye with GON according to an embodiment of the present invention. Similar to the segmentation processes illustrated in FIGS. 5A and 5B, the ROI of the retinal image was segmented to provide the segmented macula images shown in FIGS. 5C and 5D.

To produce the ROIs shown in FIGS. 5A-5D, an intensity-based method was used for ROI localization. For consistency of the process for detection of the optic disc and the macula, the background brightness was corrected by normalizing the intensity of various images beforehand. With the retina pattern, the optic disc illustrated in FIGS. 5A and 5B shows the highest intensity, while the fovea (i.e., the center of the macula illustrated in FIGS. 5C and 5D shows lower intensity in fundus images). The probability density function (PDF) peak was defined as the highest intensity value in the histogram of the optic disc. Then, the pixels were set to the optic disc region. Accordingly, we could locate the macular region with the lowest intensity values by computing the intensity values that were within the 99% confidence interval (CI) of the PDF corresponding to the center of mass. The center of the macula is illustrated by the square near the center of the segmented images shown in FIGS. 5C and 5D. After the segmentation, the retinal analysis data shown in the tables herein was computed, for example, using the ARIA method.

There are two ROIs are illustrated in FIGS. 5A-5D. Additionally, in some embodiments, the original retinal image is utilized by the methods and systems described herein. One of ordinary skill in the art would recognize many variations, modifications, and alternatives.

All the images used for training and testing were obtained at the Department of Ophthalmology, Zhongshan Hospital of Fudan University. As discussed above in relation to FIG. 2, we applied a transfer net ResNet-50 deep network and the ARIA automatic features generation approach to generate features and used the Glmnet approach to select the associated features. Then, we applied 10-fold cross-validation with RF bagtree models in the primary dataset and finally confirmed the estimation performance of our RF models in the validation dataset. The estimation performance in quantifying glaucomatous parameters was evaluated by calculating the root-mean-square error (RMSE), the mean absolute error (MAE), and the Pearson correlation coefficient.

In our ensemble RF bagtree models, we obtained a Pearson correlation coefficient of 0.640, RMSE of 11.998, and MAE of 9.096 mm in 10-fold cross-validation and Pearson correlation coefficient of 0.466, RMSE of 13.834, and MAE of 10.783 mm in the validation dataset.

A total of 1131 images paired with OCT optic disc scans and 1021 images paired with OCT macular scans from 544 patients were included as the primary dataset. The validation dataset consisted of 269 images from 130 patients. In both the primary dataset and validation dataset, there were significant correlations between estimated and true values in all OCT parameters (p<0.001) including RNFL thickness (correlation coefficient r=0.640 and r=0.466), rim area (r=0.624 and r=0.385), disc area (r=0.480 and r=0.330), average C/D (r=0.648 and r=0.511), vertical C/D (r=0.678 and r=0.529), cup volume (r=0.578 and r=0.443), average GCIPL thickness (r=0.583 and r=0.368), and minimum GCIPL thickness (r=0.557 and r=0.344), respectively.

The database contained information on medical history and comprehensive ophthalmologic examinations including non-mydriatic color fundus retinal images (TOPCON TRC-NW100 Non-Mydriatic Retinal Camera, Tokyo, Japan), Cirrus HD-OCT (Carl Zeiss Meditec 5000, Dublin, CA, USA), and Humphrey Field Analyzer (HFA, Carl Zeiss Meditec, 750i, Dublin, CA, USA). Cirrus HD-OCT optic disc scan automatically locates a circle of the 3.46 mm diameter evenly around the center of the optic disc and generates 200×200 Optic Disc Cube data through a 6 mm square grid. Cirrus HD-OCT macular scan includes the Ganglion Cell OU Analysis measures the thicknesses for the sum of the ganglion cell layer and inner plexiform layer (GCL+IPL) using data from the 512×128 Macular Cube centered on the fovea and generates a cube of data through a 6 mm square grid.

Besides retinal images, we also collected patient's medical history and OCT reports. Each image was paired with the corresponding OCT image, which date is the closest to the date of the image. The inclusion criteria of subjects were 1) age equal to or older than 18 years old, 2) gradable color fundus retinal images, 3) reliable OCT scans, and 4) the interval between the dates of images and the date of corresponding OCT less than 1 month. The exclusion criteria were 1) other ocular or systemic diseases that may affect the optic nerve or 2) missing data on OCT scans. Ungradable image quality was defined as optic disc or macular vessels invisible on the surface. Unreliable OCT reports were defined as inaccurate data resulting from apparent eye movements, involuntary blinking or saccade, or those with a signal strength index <6 were excluded, as recommended by the manufacturer.

All the images with the paired OCT optic disc scans composed dataset A to estimate RNFL thickness and ONH parameters, including rim area, disc area, average C/D, vertical C/D, and cup volume on color fundus retinal images from the OCT optic disc scan. All the images with the paired OCT macular scans composed dataset B to estimate average GCIPL thickness and minimum GCIPL thickness from the OCT macular scan.

We labeled all the images in primary datasets A and B with:
1. images without GON;
2. images with probable GON; and
3. images with definite GON.

Two ophthalmologists with three years of clinical experience in glaucoma assessed GON based on pairs of gradable images and reliable OCT reports based on the definition of classification shown in Table 1. If there were a discordant result between ophthalmologists, another specialist with over ten years of glaucoma experience would make a final decision.

TABLE 1
The definition of GON classification

| Classification | Definition |
|---|---|
| No GON | No signs of below conditions |
| Suspect GON | At least 2 conditions in: 1. 0.7 ≤ VCDR < 0.85; 2. rim width ≤0.1DD; |
| | 3. general rim thinning ≥60° or localized rim thinning <60°(11 to 1 |
| | o'clock or 5 to 7 o'clock); 4. RNFL defects; 5. splinter hemorrhages; |
| | and 6. peripapillary atrophy (β zone) |
| Definite GON | At least 1 condition in: 1. VCDR ≥0.85; 2. RNFL defects correspond |
| | with the thinning area of rim or notches |

A validation dataset was also collected from the Department of Ophthalmology, Zhongshan Hospital of Fudan University. Additionally, the repository contained medical history, OCT (Cirrus, Carl Zeiss 5000), and visual field test (Humphrey automated perimetry). Besides the inclusion and exclusion of the primary dataset, subjects were also excluded if the visual field test had more than 33% fixation losses or more than 15% false-positive errors.

The images were matched with an OCT optic disc scan, an OCT macular scan, and a reliable VF test. A validation dataset with OCT optic disc and macular scans were used to confirm ARIA's performance further to assess RNFL thickness, ONH parameters, and GCIPL thickness on unseen images. The patients in the validation dataset have no overlap with the primary dataset to test the generalization of the methods described herein. There were three groups in the validation dataset: 1. control (health); 2. pre-perimetric glaucoma (PPG); and 3. glaucoma based on the color fundus retinal images, OCT ONH scans, and VF test with the details of classification. Adding the VF test can provide functional glaucomatous defect more than structure defect for further analysis stratified by different groups. Preperimetric glaucoma (PPG) was defined as the presence of characteristic glaucomatous changes without the presence of VF defects. Glaucoma was defined as the presence of characteristic glaucomatous changes with the corresponding presence of VF defects. The definition of glaucomatous VF defect is (1) a cluster of 3 points with probabilities <5% in at least one hemifield on the pattern deviation map, including at least 1 point with a probability <1% or a cluster of 2 points with a probability <1%; (2) outside of the normal limits on glaucoma hemifield test; or (3) a pattern standard deviation <5%.

The performance of the RF bagtree models in quantifying glaucomatous damage on images was evaluated by calculating the R square, the root-mean-square error (RMSE), and the mean absolute error (MAE) to evaluate the prediction accuracy and the difference. We also calculated the Pearson correlation coefficient to estimate the agreement between observed OCT parameters and predicted values of algorithms. For the demographic data of patients, one-way ANOVA was used to compare continuous data and Chi-square tests were used for categorical data. P values <0.05 were considered statistically significant.

As discussed above, the primary dataset A consisted of 1131 images paired with OCT optic disc scans and the primary dataset B consisted of 1021 images paired with OCT macular scans. Additional details related to these datasets is provided in Table 2 and Table 3. Overall, there were 544 patients (190 without GON, 174 with probable GON, and 180 with definite GON) included in our primary dataset. Age is significantly different in the three groups of patients (p<0.001), whereas sex has no significant difference (p=0.051).

TABLE 2
Demographic data of patients and images in the primary datasets

| | No GON | Probable GON | Definite GON |
|---|---|---|---|
| | Mean ± SD | | |
| Patients | | | |
| Number | 190 | 174 | 180 |
| Age | 48.3 ± 15.9 | 52.4 ± 18.2 | 62.9 ± 15.4 |
| Sex (Female) | 55.8% | 47.7% | 43.3% |
| Images in dataset A | | | |
| Number | 504 | 359 | 268 |
| RNFL thickness (μm) | 97.91 ± 8.88 | 87.86 ± 12.04 | 69.06 ± 14.43 |
| Rim area (mm²) | 1.33 ± 0.24 | 1.13 ± 0.28 | 0.80 ± 0.27 |
| Disc area (mm²) | 2.01 ± 0.40 | 2.15 ± 0.51 | 2.16 ± 0.52 |
| Average C/D | 0.54 ± 0.16 | 0.65 ± 0.15 | 0.78 ± 0.10 |
| Vertical C/D | 0.51 ± 0.15 | 0.62 ± 0.15 | 0.78 ± 0.09 |
| Cup volume (mm³) | 0.22 ± 0.16 | 0.39 ± 0.26 | 0.56 ± 0.28 |
| Images in dataset B | | | |
| Number | 464 | 331 | 226 |
| Average GCIPL | 81.33 ± 7.88 | 75.59 ± 10.35 | 62.56 ± 11.39 |
| Thickness (μm) | | | |
| Minimum GCIPL | 76.03 ± 13.46 | 68.95 ± 16.50 | 49.27 ± 16.31 |
| Thickness (μm) | | | |

The prediction performance of models in datasets A and B in RF bagtree models for 10-fold cross-validation are shown in Table 3. The interpretation of correlation coefficients of 0.00-0.10, 0.11-0.39, 0.40-0.69, 0.70-0.89, and 0.90-1.00 were suggested for the negligible, weak, moderate, strong, and very strong correlation, respectively. Our algorithms showed significant (p<0.001) and moderate correlations between the predicted values of algorithms and true values of OCT scans for all the OCT parameters. Additionally, our algorithm to estimate vertical C/D showed the highest correlation coefficient of 0.678 with the R² of 0.46, RMSE of 0.127, and MAE of 0.092, followed by vertical C/D with an correlation coefficient of 0.648, the R² of 0.42, RMSE of 0.128, and MAE of 0.093. The model's performance for disc area prediction showed the weakest correlation with a coefficient of 0.480, a R² of 0.23, an RMSE of 0.411, and an MAE of 0.311.

TABLE 3
The prediction performance of ensemble RF bagtree models
in 10-fold cross-validation in the primary datasets

| | | | | |
|---|---|---|---|---|
| R² | RMSE | MAE | r | p |

Dataset A

| | | | | | |
|---|---|---|---|---|---|
| RNFL thickness (μm) | 0.41 | 11.998 | 9.096 | 0.640 | <0.001 |
| Rim area (mm²) | 0.39 | 0.253 | 0.190 | 0.624 | <0.001 |
| Disc area (mm²) | 0.23 | 0.411 | 0.311 | 0.480 | <0.001 |
| Average C/D | 0.42 | 0.128 | 0.093 | 0.648 | <0.001 |
| Vertical C/D | 0.46 | 0.127 | 0.092 | 0.678 | <0.001 |
| Cup volume (mm³) | 0.31 | 0.214 | 0.167 | 0.557 | <0.001 |

Dataset B

| | | | | | |
|---|---|---|---|---|---|
| Average GCIPL | 0.34 | 9.624 | 7.007 | 0.583 | <0.001 |
| thickness (μm) | | | | | |
| Minimum GCIPL | 0.31 | 14.677 | 10.358 | 0.557 | <0.001 |
| thickness (μm) | | | | | |

As discussed above, the validation dataset consisted of 269 images paired with OCT optic disc scans, OCT macular scans, and VF tests, including 136 with no glaucoma, 52 with PPG, and 81 with glaucoma from 130 patients. This validation dataset is illustrated in Table 4. Age is also significantly different in the three groups of patients (p<0.001) whereas sex has no significant difference (p=0.436).

Table 5 shows the prediction performance of the ensemble RF bagtree models in the validation dataset. The column labeled “True” corresponds to the average OCT measurement for the various OCT parameters for all patients in the validation dataset (i.e., n=269 patients). The column labeled “Estimated” corresponds to the estimated values for the various OCT parameters for all patients in the validation dataset In the estimation performance of RF models in the validation dataset shown in Table 5, all the OCT parameters also showed a significant correlation with the predicted values (p<0.001). Similarly, our algorithms also performed best and showed a moderate correlation in vertical C/D and average C/D with a correlation coefficient of 0.529, a RMSE of 0.130, and a MAE of 0.098, and a correlation coefficient of 0.511, a RMSE of 0.130, and a MAE of 0.100, respectively. Similarly, the model's performance for disc area prediction showed the weakest correlation with a coefficient of 0.330, a R² of 0.23, a RMSE of 0.411, and a MAE of 0.311.

TABLE 4
The demographic data in the validation dataset (n = 269)

| | | |
|---|---|---|
| | Preperimetric | |
| Control | glaucoma (PPG) | Glaucoma |

Mean ± SD

Patients

| | | | |
|---|---|---|---|
| Number | 63 | 19 | 48 |
| Age | 38.7 ± 13.0 | 39.8 ± 15.1 | 54.4 ± 15.1 |
| Sex (Female) | 54.0% | 47.4% | 41.7% |

Images

| | | | |
|---|---|---|---|
| Number | 136 | 52 | 81 |
| RNFL thickness | 96.81 ± 8.98 | 90.62 ± 9.82 | 74.63 ± 13.40 |
| (μm) | | | |
| Rim area (mm²) | 1.23 ± 0.25 | 1.05 ± 0.25 | 0.88 ± 0.23 |
| Disc area (mm²) | 1.95 ± 0.37 | 2.24 ± 0.68 | 2.22 ± 0.64 |
| Average C/D | 0.57 ± 0.13 | 0.70 ± 0.13 | 0.75 ± 0.08 |
| Vertical C/D | 0.54 ± 0.12 | 0.67 ± 0.12 | 0.76 ± 0.10 |
| Cup volume | 0.24 ± 0.14 | 0.58 ± 0.36 | 0.55 ± 0.32 |
| (mm³) | | | |
| Average GCIPL | 82.17 ± 5.65 | 77.15 ± 8.34 | 68.46 ± 12.35 |
| Thickness (μm) | | | |
| Minimum GCIPL | 79.33 ± 7.40 | 74.67 ± 8.18 | 58.65 ± 16.33 |
| Thickness (μm) | | | |
| VFI (%) | 98.90 ± 1.43 | 98.54 ± 1.26 | 75.11 ± 29.41 |
| MD (dB) | −0.84 ± 4.67 | −1.57 ± 1.38 | −9.51 ± 8.92 |
| PSD (dB) | 1.79 ± 0.61 | 1.94 ± 0.67 | 6.62 ± 3.97 |

TABLE 5
Prediction performance of ensemble RF bagtree
models in the validation dataset (n = 269)

| | True | Estimated | RMSE | MAE | r | P |
|---|---|---|---|---|---|---|
| RNFL thickness | 88.93 ± 14.37 | 84.17 ± 9.48 | 13.834 | 10.783 | 0.466 | <0.001 |
| (μm) | | | | | | |
| Rim area (mm²) | 1.09 ± 0.29 | 1.07 ± 0.23 | 0.291 | 0.228 | 0.385 | <0.001 |
| Disc area (mm²) | 2.09 ± 0.55 | 1.99 ± 0.12 | 0.530 | 0.378 | 0.330 | <0.001 |
| Average C/D | 0.65 ± 0.14 | 0.62 ± .10 | 0.130 | 0.100 | 0.511 | <0.001 |
| Vertical C/D | 0.63 ± 0.15 | 0.61 ± 0.10 | 0.130 | 0.098 | 0.529 | <0.001 |
| Cup volume | 0.40 ± 0.30 | 0.36 ± 0.08 | 0.275 | 0.184 | 0.443 | <0.001 |
| (mm³) | | | | | | |
| Average GCIPL | 77.07 ± 10.51 | 69.07 ± 4.99 | 12.670 | 10.567 | 0.368 | <0.001 |
| thickness (μm) | | | | | | |
| Minimum GCIPL | 72.20 ± 14.23 | 57.51 ± 4.59 | 19.841 | 18.083 | 0.344 | <0.001 |
| thickness (μm) | | | | | | |

In addition, we conducted subsidiary analyses in the subgroups of control, PPG, and glaucoma as shown in Table 6. For clarity, Table 6 is presented in three sets of columns, Table 6A, Table 6B, and Table 6C, which together form Table 6 and provide values for the RNFL thickness (μm), Rim area (mm²), Disc area (mm²), Average C/D, Vertical C/D, Cup volume (mm₃), Average GCIPL thickness (μm), and Minimum GCIPL thickness (μm) for the Control group, the PPG group, and the Glaucoma group, respectively, i.e., the prediction performance of ensemble RF bagtree models in the validation dataset stratified by glaucoma groups.

TABLE 6A
Prediction performance of ensemble RF bagtree models
in the validation dataset for the Control group

| | Control | | | |
|---|---|---|---|---|
| | True | Estimated | r | P |
| RNFL thickness | 96.80 ± 8.98 | 88.77 ± 7.60 | 0.328 | <0.001** |
| (μm) | | | | |
| Rim area (mm²) | 1.23 ± 0.25 | 1.18 ± 0.19 | 0.179 | 0.037* |
| Disc area (mm²) | 1.95 ± 0.37 | 1.95 ± 0.11 | 0.139 | 0.106 |
| Average C/D | 0.57 ± 0.13 | 0.57 ± 0.10 | 0.299 | <0.001** |
| Vertical C/D | 0.536 ± 0.12 | 0.56 ± 0.10 | 0.278 | 0.001** |
| Cup volume | 0.32 ± 0.07 | 0.24 ± 0.14 | 0.328 | <0.001** |
| (mm³) | | | | |
| Average GCIPL | 82.17 ± 5.65 | 71.25 ± 4.18 | 0.115 | 0.181 |
| thickness (μm) | | | | |
| Minimum GCIPL | 79.33 ± 7.40 | 59.35 ± 4.02 | 0.079 | 0.360 |
| thickness (μm) | | | | |

*<0.05;
**<0.01

TABLE 6B
Prediction performance of ensemble RF bagtree models
in the validation dataset for the PPG group

| | PPG | | | |
|---|---|---|---|---|
| | True | Estimated | r | P |
| RNFL thickness | 90.62 ± 9.82 | 80.71 ± 8.85 | −0.021 | 0.882 |
| (μm) | | | | |
| Rim area (mm²) | 1.05 ± 0.25 | 0.98 ± 0.21 | −0.116 | 0.412 |
| Disc area (mm²) | 2.24 ± 0.68 | 1.03 ± 0.13 | 0.527 | <0.001** |
| Average C/D | 0.70 ± 0.13 | 0.66 ± 0.10 | 0.325 | 0.019* |
| Vertical C/D | 0.67 ± 0.12 | 0.65 ± 0.10 | 0.331 | 0.017* |
| Cup volume | 0.40 ± 0.08 | 0.58 ± 0.36 | 0.322 | 0.016* |
| (mm³) | | | | |
| Average GCIPL | 77.15 ± 8.34 | 67.39 ± 4.77 | −0.142 | 0.31 |
| thickness (μm) | | | | |
| Minimum GCIPL | 74.67 ± 8.18 | 55.96 ± 4.63 | 0.034 | 0.812 |
| thickness (μm) | | | | |

*<0.05;
**<0.01

TABLE 6C
Prediction performance of ensemble RF bagtree models
in the validation dataset for the Glaucoma group

| | Glaucoma | | | |
|---|---|---|---|---|
| | True | Estimated | r | P |
| RNFL thickness | 74.63 ± 13.40 | 78.66 ± 8.89 | 0.323 | <0.001** |
| (μm) | | | | |
| Rim area (mm²) | 0.88 ± 0.23 | 0.95 ± 0.21 | 0.425 | <0.001** |
| Disc area (mm²) | 2.22 ± 0.64 | 2.02 ± 0.10 | 0.182 | 0.104 |
| Average C/D | 0.75 ± 0.10 | 0.68 ± .10 | 0.405 | <0.001** |
| Vertical C/D | 0.76 ± 0.10 | 0.66 ± 0.09 | 0.403 | <0.001** |
| Cup volume | 0.40 ± 0.07 | 0.55 ± 0.32 | 0.223 | 0.045* |
| (mm³) | | | | |
| Average GCIPL | 68.46 ± 12.35 | 66.47 ± 4.77 | 0.377 | <0.001** |
| thickness (μm) | | | | |
| Minimum GCIPL | 58.65 ± 16.22 | 55.43 ± 4.26 | 0.319 | 0.004** |
| thickness (μm) | | | | |

*<0.05;
**<0.01

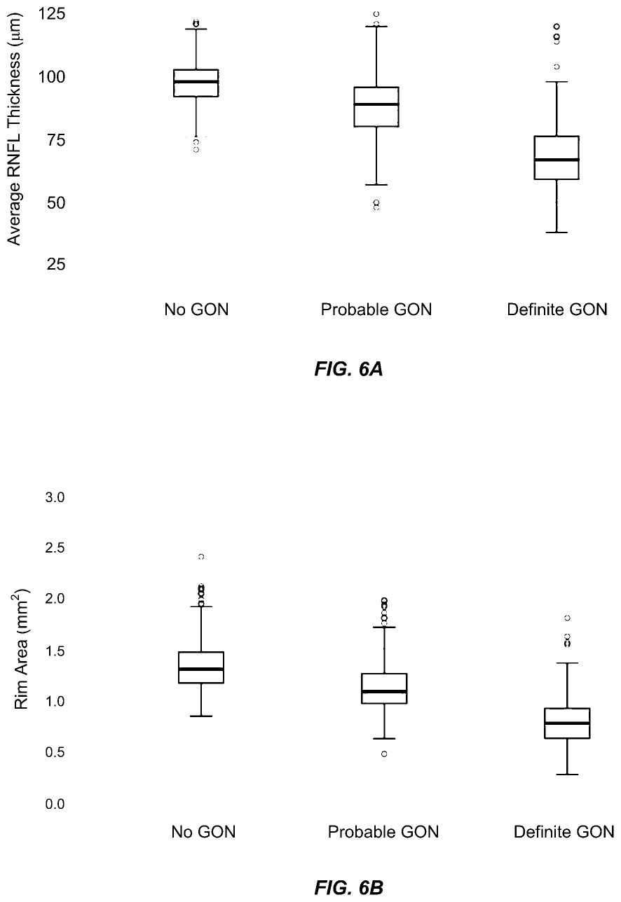

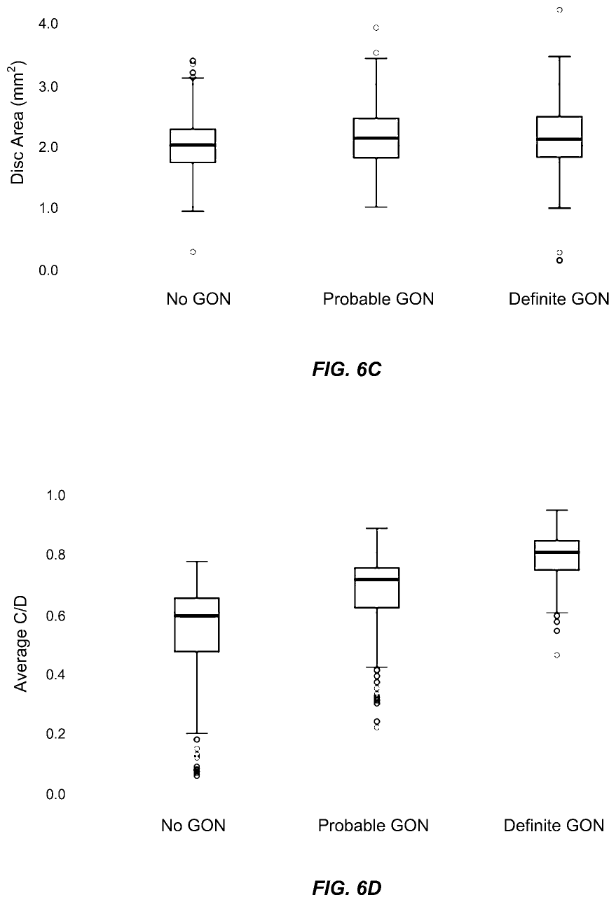

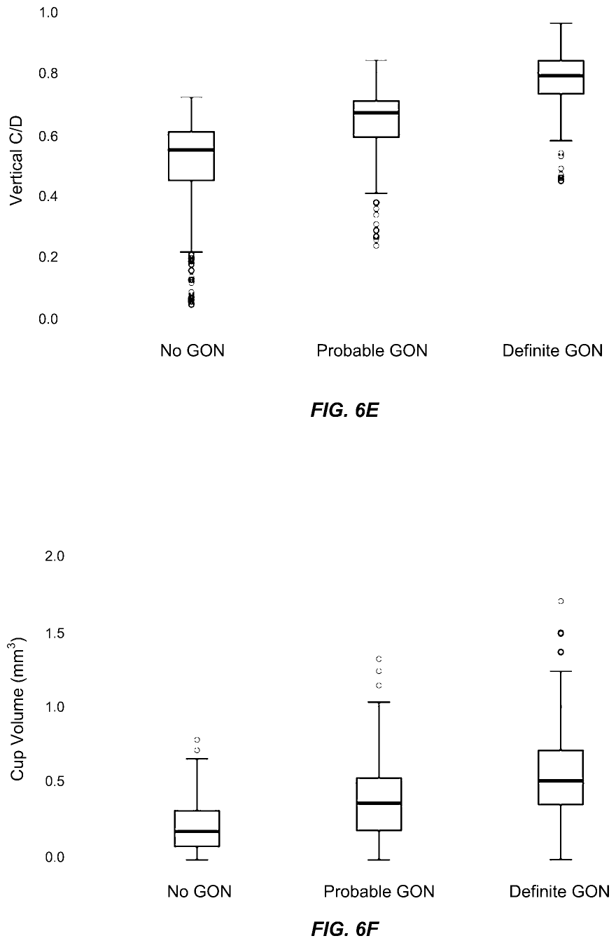

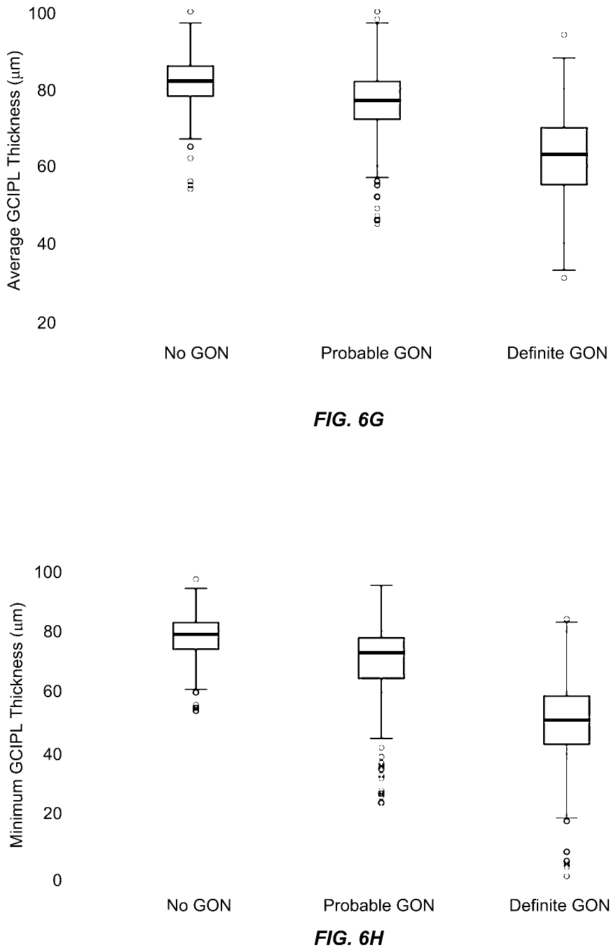

FIGS. 6A-6H are box plots of OCT parameters according to an embodiment of the present invention. The data presented in FIGS. 6A-6F was estimated using segmented optic disc images. The data presented in FIGS. 6G-6H was estimated using segmented macula images.

FIG. 6A illustrates box plots for average RNFL thickness in microns for patients without GON, with probable GON, and with definite GON. FIG. 6B illustrates box plots for rim area in square millimeters for patients without GON, with probable GON, and with definite GON. FIG. 6C illustrates box plots for disc area in square millimeters for patients without GON, with probable GON, and with definite GON. FIG. 6D illustrates box plots for average C/D for patients without GON, with probable GON, and with definite GON. FIG. 6E illustrates box plots for vertical C/D for patients without GON, with probable GON, and with definite GON. FIG. 6F illustrates box plots for cup volume in square millimeters for patients without GON, with probable GON, and with definite GON.

FIG. 6G illustrates box plots for average GCIPL thickness in microns for patients without GON, with probable GON, and with definite GON. FIG. 6H illustrates box plots for minimum GCIPL thickness in microns for patients without GON, with probable GON, and with definite GON.

OCT parameters were estimated better in the glaucoma group than in the control group and PPG group. In the glaucoma group, most of the predicted values have significant correlations with true OCT values except the parameter of disc area. There are three OCT parameters that have significant correlations in all the subgroups, average C/D with p<0.001, 0.019, <0.001, vertical C/D with p=0.001, 0.017, <0.001, and cup volume with p<0.001, 0.016, and 0.045, in the groups of control, PPG, and glaucoma, respectively.

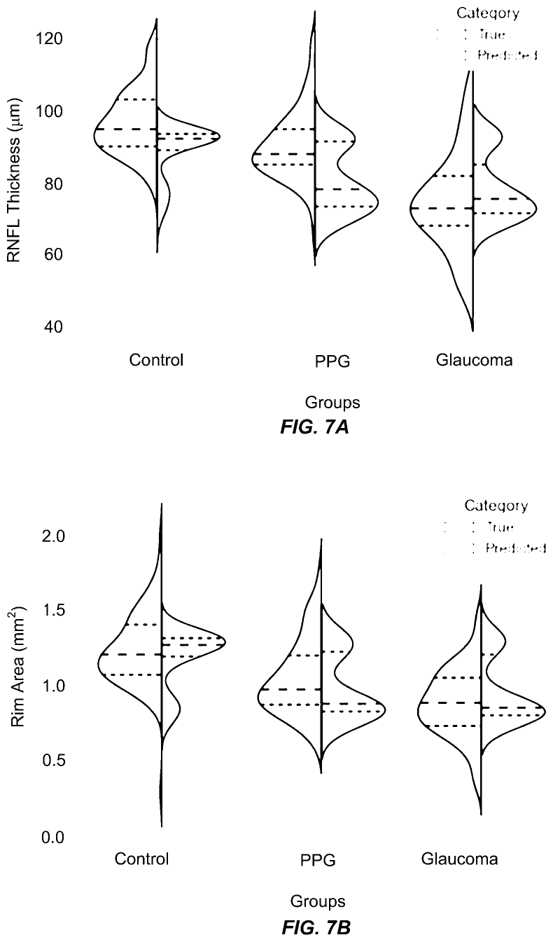

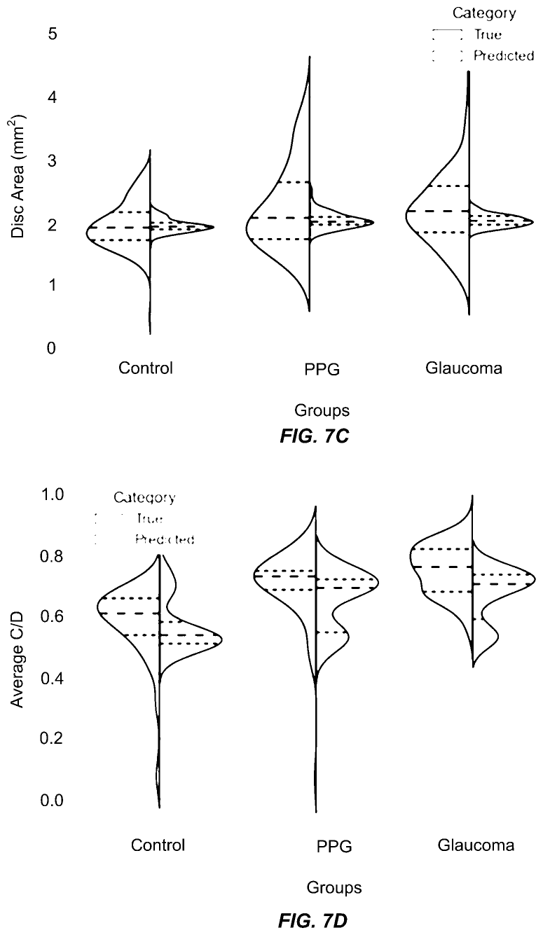

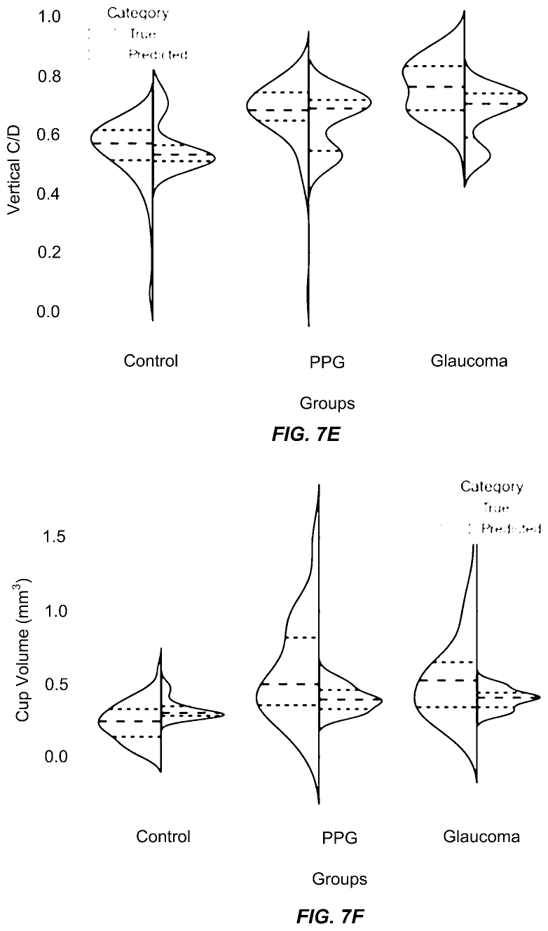

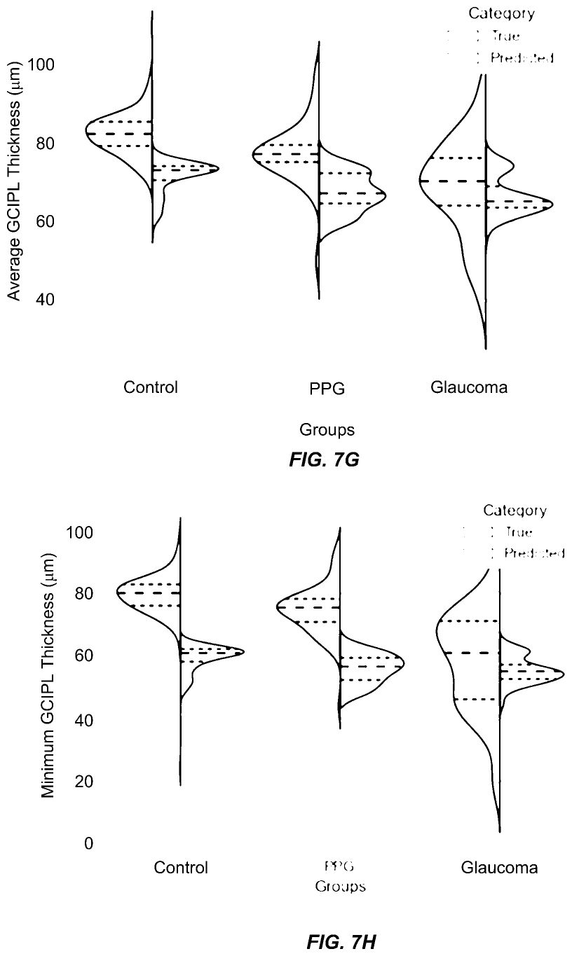

FIGS. 7A-7G are violin plots demonstrating true and estimated OCT parameters according to an embodiment of the present invention. The violin plots for each OCT parameter shown in FIGS. 7A-7G also demonstrated the relationship between the true OCT values and the estimated values of the OCT parameters estimated using the methods and systems described herein, stratified by groups in the validation dataset.

FIG. 7A illustrates violin plots for average RNFL thickness in microns for patients in the control group, the PPG group, and the glaucoma group. FIG. 7B illustrates violin plots for rim area in square millimeters for patients in the control group, the PPG group, and the glaucoma group. FIG. 7C illustrates violin plots for disc area in square millimeters for patients in the control group, the PPG group, and the glaucoma group. FIG. 7D illustrates violin plots for average C/D for patients in the control group, the PPG group, and the glaucoma group. FIG. 7E illustrates violin plots for vertical C/D for patients in the control group, the PPG group, and the glaucoma group. FIG. 7F illustrates violin plots for cup volume in square millimeters for patients in the control group, the PPG group, and the glaucoma group.

FIG. 7G illustrates violin plots for average GCIPL thickness in microns for patients in the control group, the PPG group, and the glaucoma group. FIG. 7H illustrates violin plots for minimum GCIPL thickness in microns for patients in the control group, the PPG group, and the glaucoma group.

In our study, the ARIA method showed good prediction performance and agreement in quantitatively assessing eight continuous OCT parameters. Both in 10-fold cross-validation and in the validation dataset, the OCT values estimated by ARIA showed significant correlations with the true OCT values for all the parameters. Thus, embodiments of the present invention provide methods and systems for developing and validating an automatic analysis method to estimate the comprehensive OCT parameters by assessing non-mydriatic color fundus retinal images. As a result, embodiments of the present invention, including embodiments incorporating the ARIA method, provide a convenient, cost-effective, and accurate tool to screen, diagnose, and monitor glaucoma and other various diseases that can be assessed by OCT scans.

Additionally, the comprehensive evaluation of combined RNFL thickness, ONH parameters, and GCIPL thickness, according to the methods described herein, may be able to accurately detect progressive changes in diseases over time.

The methods and systems described herein performed well in predicting average and vertical C/D with correlation coefficients of 0.648 and 0.678 in 10-fold cross-validation, and 0.511 and 0.529 in the validation dataset, respectively. This can be explained by the 2D nature of these parameters, which should be more direct and easy to estimate based on 2D fundus images.

In the validation stratified by groups, ARIA performed better in the glaucoma group than in the control group and PPG group. Therefore, our algorithms may be more beneficial to monitor glaucoma progression in patients with glaucoma. On the other hand, glaucoma severity had a significant effect on the diagnostic performance of the Cirrus HD-OCT. A study found that for the OCT parameter of average RNFL thickness, AUCs were 0.962, 0.932, 0.886, and 0.822 for visual field index (VFI) of 70%, 80%, 90%, and 100% from standard automated perimeter, respectively. Hence, the precise estimation of OCT parameters in the glaucoma group may also help to accurately detect glaucoma by providing information for both the ophthalmologists and the automatic methods.

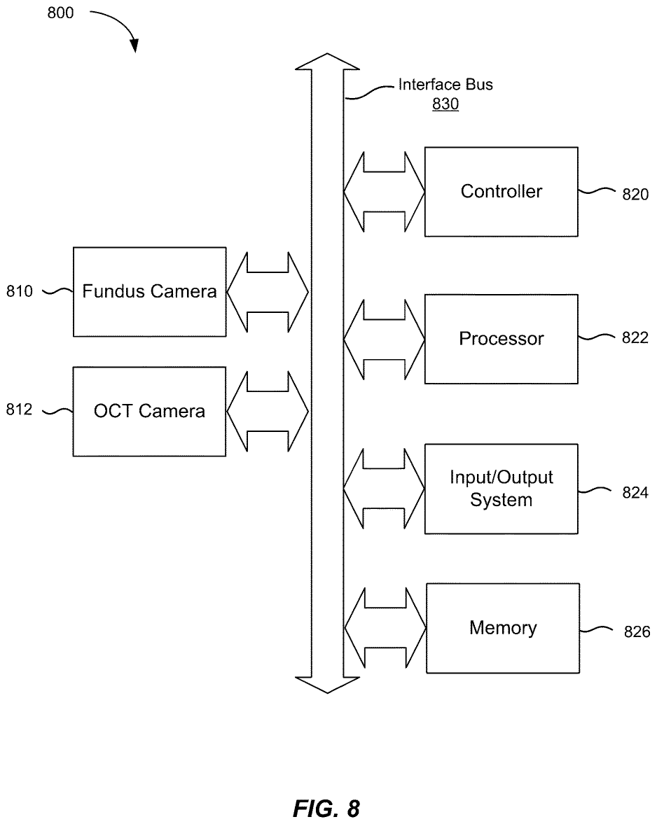

FIG. 8 is a simplified schematic diagram illustrating an OCT parameter estimation system according to an embodiment of the present invention. The OCT parameter estimation system 800 includes a fundus camera 810, which can capture color (e.g., RGB) retinal images. The OCT parameter estimation system 800 also includes an OCT camera 812 operable to capture and characterize 3D parameters corresponding to the retina.

The OCT parameter estimation system 800 further includes controller 820, processor 822, an input/output system 824, and a memory 826. Controller 820, which can be a computer controller, is utilized to operate the various system elements, for example, obtaining and processing images captured using the fundus camera 810 and OCT parameters obtained from the OCT camera 812. The processor 822 can perform the ARIA method described herein. Additionally, the processor 822 can implement the neural network and other processing described herein. Accordingly, the captured images are provided to processor 822, which may be a computer processor coupled to input/output system 824. The various elements of OCT parameter estimation system 800 are connected via interface bus 830, which provides for control and data signals to be transmitted to/from and received to/from one or more of the various elements.

Various examples of the present disclosure are provided below. As used below, any reference to a series of examples is to be understood as a reference to each of those examples disjunctively (e.g., “Examples 1-4” is to be understood as “Examples 1, 2, 3, or 4”).

Example 1 is a method of estimating optical coherence tomography (OCT) parameters, the method comprising: obtaining a retinal fundus image; segmenting the retinal fundus image to produce a region of interest 2D image; inputting the region of interest 2D image into a neural network; and generating estimated OCT parameters using the neural network.

Example 2 is the method of example 1 wherein the OCT parameters include retinal nerve fiber layer (RNFL) thickness.

Example 3 is the method of example(s) 1-2 wherein the RNFL thickness comprises an average RNFL thickness.

Example 4 is the method of example(s) 1-3 wherein the region of interest 2D image includes an optic disc image and the RNFL thickness is derived from an OCT optic disc scan.

Example 5 is the method of example(s) 1-4 wherein the OCT parameters include optic nerve head (ONH) parameters.

Example 6 is the method of example(s) 1-5 wherein the ONH parameters include rim area, disc area, average cup-to-disc-ratio (C/D), vertical C/D, or cup volume.

Example 7 is the method of example(s) 1-6 wherein the region of interest 2D image includes an optic disc image and the ONH parameters are derived from an OCT optic disc scan.

Example 8 is the method of example(s) 1-7 wherein the OCT parameters include ganglion cell-inner plexiform layer (GCIPL) thickness.

Example 9 is the method of example(s) 1-8 wherein the GCIPL thickness comprises an average GCIPL thickness.

Example 10 is the method of example(s) 1-9 wherein the GCIPL thickness comprises a minimum GCIPL thickness.

Example 11 is the method of example(s) 1-10 wherein the region of interest 2D image includes a macula image and the GCIPL thickness is derived from an OCT macular scan.

Example 12 is the method of example(s) 1-11 further comprising recommending a medical procedure based on the estimated OCT parameters.

Example 13 is the method of example(s) 1-12 further comprising obtaining one or more additional retinal fundus images based on the estimated OCT parameters.

Example 14 is a method for diagnosing, indicating a severity of, or predicting and monitoring a progression of one or more diseases correlated with OCT parameters, the method comprising: providing a plurality of color images; receiving a plurality of OCT data sets, each of the OCT data sets corresponding to one of the plurality of color images; training a deep learning model using the plurality of color images and the plurality of OCT data sets; providing a patient color image; providing the patient color image as an input to the deep learning model; and estimating the OCT parameters using the deep learning model.

Example 15 is the method of example 14 wherein the one or more diseases comprise neuro-ophthalmology diseases including glaucoma, nonarteritic ischemic optic neuropathy (NAION), Leber's hereditary optic neuropathy (LHON), compressive optic neuropathies, disc swelling, idiopathic intracranial hypertension, or neuro-myelitis optica (NMO) spectrum disorder.

Example 16 is the method of example(s) 14-15 wherein the one or more diseases comprise neurodegeneration diseases including Alzheimer's disease (AD), mild cognitive impairment (MCI), Parkinson's disease, dementia, Huntington's disease, autism spectrum disorder (ASD), attention deficit hyperactivity disorder (ADHD), or mental health diagnoses including depression.

Example 17 is the method of example(s) 14-16 wherein the one or more diseases comprise diseases related to one or more OCT parameters, the diseases including hypertensive retinopathy, high blood pressure, intracranial tumors that affect optic pathway, sellar and parasellar lesions, axonal degeneration in eyes of patients with multiple sclerosis (MS), diabetic peripheral neuropathy (DPN), or Type 1 diabetes.

Example 18 is the method of example(s) 14-17 wherein the one or more diseases comprise MRI-measured brain atrophy or spinal cord lesions.

Example 19 is the method of example(s) 14-18 wherein the plurality of color images comprise 2D fundus camera images.

Example 20 is the method of example(s) 14-19 wherein the plurality of color images comprise segmented region of interest images.

Example 21 is the method of example(s) 14-20 further comprising: applying statistical analysis to features corresponding to the deep learning model; and extracting a subset of the features to provide a set of significant features.

Example 22 is a system comprising: a fundus camera; a memory; and a processor coupled to the memory, wherein the processor is configured to: obtain a retinal fundus image; segment the retinal fundus image to produce a region of interest 2D image; and generate estimated OCT parameters using the processor.

Example 23 is the system of example 22 wherein the processor implements a neural network.

Example 24 is the system of example(s) 22-23 wherein the OCT parameters include retinal nerve fiber layer (RNFL) thickness.

Example 25 is the system of example(s) 22-24 wherein the RNFL thickness comprises an average RNFL thickness.

Example 26 is the system of example(s) 22-25 wherein the region of interest 2D image includes an optic disc image and the RNFL thickness is derived from an OCT optic disc scan.

Example 27 is the system of example(s) 22-26 wherein the OCT parameters include optic nerve head (ONH) parameters.

Example 28 is the system of example(s) 22-27 wherein the ONH parameters include rim area, disc area, average cup-to-disc-ratio (C/D), vertical C/D, or cup volume.

Example 29 is the system of example(s) 22-28 wherein the region of interest 2D image includes an optic disc image and the ONH parameters are derived from an OCT optic disc scan.

Example 30 is the system of example(s) 22-29 wherein the OCT parameters include ganglion cell-inner plexiform layer (GCIPL) thickness.

Example 31 is the system of example(s) 22-30 wherein the GCIPL thickness comprises an average GCIPL thickness.

Example 32 is the system of example(s) 22-31 wherein the GCIPL thickness comprises

a minimum GCIPL thickness.

Example 33 is the system of example(s) 22-32 wherein the region of interest 2D image includes a macula image and the GCIPL thickness is derived from an OCT macular scan.

Example 34 is the system of example(s) 22-33 wherein the processor is further configured to obtain one or more additional retinal fundus images based on the estimated OCT parameters.

In the foregoing specification, the disclosure has been described with reference to specific embodiments thereof. It will, however, be evident that various modifications and changes may be made thereto without departing from the broader spirit and scope of the disclosure. The specification and drawings are, accordingly, to be regarded in an illustrative rather than restrictive sense.

Indeed, it will be appreciated that the systems and methods of the disclosure each have several innovative aspects, no single one of which is solely responsible or required for the desirable attributes disclosed herein. The various features and processes described above may be used independently of one another, or may be combined in various ways. All possible combinations and subcombinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination. No single feature or group of features is necessary or indispensable to each and every embodiment.

It will be appreciated that conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Furthermore, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Accordingly, the claims are not intended to be limited to the embodiments shown herein but are to be accorded the widest scope consistent with this disclosure, the principles, and the novel features disclosed herein. Thus, it is also understood that the examples and embodiments described herein are for illustrative purposes only and that various modifications or changes in light thereof will be suggested to persons skilled in the art and are to be included within the spirit and purview of this application and scope of the appended claims.

What is claimed is:

1. A method of estimating optical coherence tomography (OCT) parameters, the method comprising:
obtaining a retinal fundus image;
segmenting the retinal fundus image to produce a region of interest 2D image;
inputting the region of interest 2D image into a neural network; and
generating estimated OCT parameters using the neural network.

2. The method of claim 1 wherein the OCT parameters include retinal nerve fiber layer (RNFL) thickness.

3. The method of claim 2 wherein the RNFL thickness comprises an average RNFL thickness.

4. The method of claim 2 wherein the region of interest 2D image includes an optic disc image and the RNFL thickness is derived from an OCT optic disc scan.

5. The method of claim 1 wherein the OCT parameters include optic nerve head (ONH) parameters.

6. The method of claim 5 wherein the ONH parameters include rim area, disc area, average cup-to-disc-ratio (C/D), vertical C/D, or cup volume.

7. The method of claim 6 wherein the region of interest 2D image includes an optic disc image and the ONH parameters are derived from an OCT optic disc scan.

8. The method of claim 1 wherein the OCT parameters include ganglion cell-inner plexiform layer (GCIPL) thickness.

9. The method of claim 8 wherein the GCIPL thickness comprises an average GCIPL thickness.

10. The method of claim 8 wherein the GCIPL thickness comprises a minimum GCIPL thickness.

11. The method of claim 8 wherein the region of interest 2D image includes a macula image and the GCIPL thickness is derived from an OCT macular scan.

12. The method of claim 1 further comprising recommending a medical procedure based on the estimated OCT parameters.

13. The method of claim 1 further comprising obtaining one or more additional retinal fundus images based on the estimated OCT parameters.

14. A method for diagnosing, indicating a severity of, or predicting and monitoring a progression of one or more diseases correlated with OCT parameters, the method comprising:
providing a plurality of color images;
receiving a plurality of OCT data sets, each of the OCT data sets corresponding to one of the plurality of color images;
training a deep learning model using the plurality of color images and the plurality of OCT data sets;
providing a patient color image;
providing the patient color image as an input to the deep learning model; and
estimating the OCT parameters using the deep learning model.

15. The method of claim 14 wherein the one or more diseases comprise neuro-ophthalmology diseases including glaucoma, nonarteritic ischemic optic neuropathy (NAION), Leber's hereditary optic neuropathy (LHON), compressive optic neuropathies, disc swelling, idiopathic intracranial hypertension, or neuro-myelitis optica (NMO) spectrum disorder.

16. The method of claim 14 wherein the one or more diseases comprise neurodegeneration diseases including Alzheimer's disease (AD), mild cognitive impairment (MCI), Parkinson's disease, dementia, Huntington's disease, autism spectrum disorder (ASD), attention deficit hyperactivity disorder (ADHD), or mental health diagnoses including depression.

17. The method of claim 14 wherein the one or more diseases comprise diseases related to one or more OCT parameters, the diseases including hypertensive retinopathy, high blood pressure, intracranial tumors that affect optic pathway, sellar and parasellar lesions, axonal degeneration in eyes of patients with multiple sclerosis (MS), diabetic peripheral neuropathy (DPN), or Type 1 diabetes.

18. The method of claim 14 wherein the one or more diseases comprise MRI-measured brain atrophy or spinal cord lesions.

19. The method of claim 14 wherein the plurality of color images comprise 2D fundus camera images.

20. The method of claim 14 wherein the plurality of color images comprise segmented region of interest images.

21. The method of claim 14 further comprising:
applying statistical analysis to features corresponding to the deep learning model; and
extracting a subset of the features to provide a set of significant features.

22. A system comprising:
a fundus camera;
a memory; and
a processor coupled to the memory, wherein the processor is configured to:
obtain a retinal fundus image;
segment the retinal fundus image to produce a region of interest 2D image; and
generate estimated OCT parameters using the processor.

23. The system of claim 22 wherein the processor implements a neural network.

24. The system of claim 22 wherein the OCT parameters include retinal nerve fiber layer (RNFL) thickness.

25. The system of claim 24 wherein the RNFL thickness comprises an average RNFL thickness.

26. The system of claim 24 wherein the region of interest 2D image includes an optic disc image and the RNFL thickness is derived from an OCT optic disc scan.

27. The system of claim 22 wherein the OCT parameters include optic nerve head (ONH) parameters.

28. The system of claim 27 wherein the ONH parameters include rim area, disc area, average cup-to-disc-ratio (C/D), vertical C/D, or cup volume.

29. The system of claim 27 wherein the region of interest 2D image includes an optic disc image and the ONH parameters are derived from an OCT optic disc scan.

30. The system of claim 22 wherein the OCT parameters include ganglion cell-inner plexiform layer (GCIPL) thickness.

31. The system of claim 30 wherein the GCIPL thickness comprises an average GCIPL thickness.

32. The system of claim 30 wherein the GCIPL thickness comprises a minimum GCIPL thickness.

33. The system of claim 30 wherein the region of interest 2D image includes a macula image and the GCIPL thickness is derived from an OCT macular scan.

34. The system of claim 22 wherein the processor is further configured to obtain one or more additional retinal fundus images based on the estimated OCT parameters.
